(19) United States
(12) Patent Application Publication
(10) Pub. No.: US 2013/0150384 A1
(43) Pub. Date: Jun. 13, 2013

(54) METHOD FOR TREATMENT OF BRAIN CANCERS

(71) Applicant: KOREA INSTITUTE OF SCIENCE AND TECHNOLOGY, Seoul (KR)

(72) Inventors: Justin Changjoon LEE, Seoul (KR); Eun-Joo Roh, Seoul (KR); Jin-Kyu Choi, Pohang-shi (KR); Kyung-Suk Han, Incheon (KR); Jung-Eun Park, Euijeongbu-shi (KR); Sang-Soo Kang, Daegu (KR); Bo Mi Ku, Jinju-si (KR); Yeon Kyung Lee, Changwon-si (KR); Antoine G. Almonte, Birmingham, AL (US); Dongho Woo, Seoul (KR); Jung-Hwan Park, Seoul (KR); Seon Ha Paek, Seoul (KR)

(73) Assignee: KOREA INSTITUTE OF SCIENCE AND TECHNOLOGY, Seoul (KR)

(21) Appl. No.: 13/768,022

(22) Filed: Feb. 15, 2013

Related U.S. Application Data
(63) Continuation of application No. 12/865,127, filed on Nov. 19, 2010, now abandoned.

(30) Foreign Application Priority Data
Jan. 31, 2008 (KR) 10-2008-0010155

(51) Int. Cl. A61K 31/522

(52) U.S. Cl. CPC A61K 31/522
USPC 514/263.34

(57) ABSTRACT

A method for preventing and/or treating brain tumor, including administering caffeine and/or its analog, and/or their pharmaceutically acceptable salt, as an active ingredient, to a patient in need thereof, is provided. The method for preventing or treating brain tumor has an activity to inhibit invasion, migration, and proliferation of brain tumor cells, and thereby very effective for the prevention and treatment of brain tumor.

### CROSS REFERENCE TO RELATED APPLICATION

This application is a Continuation application of pending U.S. Ser. No. 12/865,127 filed on Nov. 19, 2010, which is the National Stage Application of International Application No. PCT/KR2008/000602 filed on Jan. 31, 2008, which claims priority to Korean Patent Application No. 10-2008-0010155 filed on Jan. 31, 2008, the contents of which are incorporated herein by reference.

### BACKGROUND OF THE INVENTION

(a) Field of the Invention

A method for preventing and/or treating brain tumor, including administering caffeine and/or its analog, and/or their pharmaceutically acceptable salt, as an active ingredient, to a patient in need thereof, is provided.

(b) Description of the Related Art

The term “brain tumor (or cancer)” refers both to a primary brain tumor, which originates in the brain tissue or meninges covering the brain, and a secondary brain tumor, which has metastasized from skull or other area of the body. Brain tumor is distinguished from tumors occurring in other organs of the body in many aspects. First, incidence of cancers in the lungs, the stomach, and the breast are limited to one or two specific types of cancers per a given organ, and traits of such cancers tend to be shared with each other. However, kinds of tumors found in the brain are very various: some examples of brain tumors may include but not be limited to: glioblastoma multiforme, malignant glioma, lymphadenoma, germ cell tumor, metastatic tumor, and the like. Among these kinds of brain tumor, glioma, in particular Glioblastoma Multiforme (GBM), is most fatal, because it is most malignant and invasive, and has a very poor prognosis, with a median survival time of only one year or short after diagnosis. Complete surgical removal of GBM is very unlikely due to vagueness of boundary between brain and tumor. In light of the above, a strong demand exists for development of chemical treatments for GBM, beyond surgical treatments. Since no effective chemical treatments have been developed up to date, however, further researches and developments are required for in this field.

### SUMMARY OF THE INVENTION

Here, an object of the present invention is to provide techniques for effectively preventing and/or treating a brain tumor, specifically glioma including GBM, by inhibiting invasion and migration, as well as proliferation, of tumor cells.

### BRIEF DESCRIPTION OF THE DRAWINGS

FIGS. 1a through 1e show Ca²⁺ responses by various agonists for GPCR (G-protein coupled receptors) and RTK (receptor tyrosine kinases), wherein

FIG. 1a shows representative pseudo color fluorescence intensity images (380 or 340 excitation, 510 emission, above) and ratio images (below) of Fura-2 AM (5 μM) loaded glioblastoma cells before and after EGF stimulation,

FIG. 1b shows traces obtained from Ca²⁺ imaging recordings performed for four glioblastoma cell lines,

FIG. 1c shows changes in Fura-2 AM intensity ratio caused by various agonists on Fura-2 loaded U178MG cells,

FIG. 1d is a low power view of glioblastoma cells, where the upper left side is X200 image, and

FIG. 1e shows decay kinetics of Ca²⁺ release showing that GPCR and RTK agonists induce intracellular Ca²⁺ increase in human glioblastoma cells.

FIGS. 2a through 2f show results related Ca²⁺ signaling, wherein

FIG. 2a shows that decay kinetics of Ca²⁺ release in Fura-2 loaded U178MG cells, in which the Fura-2 loaded U178MG cells were stimulated with bradykinin in 2 mM Ca²⁺ HEPES buffer, Ca²⁺-free HEPES buffer, and in the presence of SKF 96365, respectively,

FIGS. 2b and 2c show decay kinetics of the representative average trace and half width(s) for each sample,

FIG. 2d shows changes in decay kinetics showing pre-treatment of U73122 blocked bradykinin- or EGF-induced Ca²⁺ release rescued by pre-treatment of U73343,

FIG. 2e shows the results of GPCR agonist application after depletion of Ca²⁺ by thapsigargin in endoplasmic reticulum, and

FIG. 2f shows decay kinetics of IP3R mediated Ca²⁺ release by bradykinin on U178MG, in the presence of ryanodine receptor antagonist.

FIGS. 3a through 3c show inhibiting activity of caffeine against migration and invation of glioblastoma, wherein

FIG. 3a shows wound area and percentage of wound closure,

FIG. 3b shows representative pictures of cells that invaded through Matrigel with varying concentrations of caffeine (top), and percentages of invaded cells (bottom), and

FIG. 3c shows representative photographs of colonies with varying concentrations of caffeine (top), and percentage of colonies numbers (bottom).

FIGS. 4a through 4h show caffeine's inhibiting activity against Ca²⁺ release, wherein

FIGS. 4a and 4b show block against intracellular Ca²⁺ release by caffeine on U178MG stimulated with bradykinin or EGF, respectively,

FIG. 4c shows % block of various agonists induced Ca²⁺ release in the presence of caffeine on U178MG,

FIG. 4d shows the inhibition level against TFLLR induced Ca²⁺ responses in the presence of caffeine at various concentrations,

FIG. 4e shows a dose response curve of Ca²⁺ release evoked by TFLLR,

FIGS. 4f and 4g show block against intracellular Ca²⁺ release by caffeine on human Glioblastoma and HEK293 stimulated with GPCR agonist, respectively, and

FIG. 4h shows % block of GPCR agonist-induced Ca²⁺ release in the presence of caffeine on each cells.

FIGS. 5a through 5d show mRNA expression rates of IP3Rs subtypes, wherein

FIG. 5a is an electophoresis image showing IP3R and GAPDH mRNA expression in human glioma cell lines (U87MG, U178MG, U373MG, T98G, M059K), human neuroblastoma cell line (SH-SY5Y), and HEK293T cell line.

FIG. 5b shows co-relation between degree of expression of IP3R subtype 3 and block of caffeine in each cell line,

FIG. 5c is an electophoresis image showing IP3R subtype mRNA expression in normal human brain cells and in human glioblastoma cells, and

FIG. 5d shows densitomeric histograms of IP3R mRNA expression in human samples.

FIGS. 6a through 6g show that caffeine specifically acts on IP₃R3, wherein

FIGS. 6a and 6b show blocks against TFLLR induced Ca² release by caffeine in HEK293T cells transfected with IP₃R1 (Bovine) and IP₃R3 (Bovine), respectively,

FIG. 6c shows % blocks by caffeine in HEK293T cells transfected with IP₃R1 (Bovine), IP₃R3 (Bovine), and IP₃R3 (Mouse),

FIG. 6d shows Ca²⁺ imaging results for the cases that only GFP was expressed and that IP3R3-shRNA plus GFP were expressed, in U178MG cells, after treating with caffeine,

FIG. 6e shows Ca²⁺ response with treating with caffeine in the control group and shRNA expression group, and

FIGS. 6f and 6g show live imaging results for cell migration of U178MG cells, with and without caffeine treatment.

FIGS. 7a through 7d show caffeine's activity in inhibiting invasion and improving viability, wherein

FIG. 7a is photographs showing U178MG cells placed on surface of 6 day aged-organotypic hippocampal slice cultures (OHSCs), in the presence or absence of caffeine,

FIG. 7b is a graph showing that invasion and migration of gliobalstoma cells in OHSCs were inhibited by caffeine.

FIG. 7c is a graph showing relative decrease in tumor size by treating caffeine, and

FIG. 7d is a graph showing increases in survival rates by caffeine intake in a brain tumor animal model.

FIG. 8 is a MTT assay result showing survival rates at various concentrations of caffeine in respective cell line.

FIGS. 9a through 9d show that caffeine action is independent upon store-operated channels or store depletion, wherein

FIG. 9a shows behaviors of Ca²⁺ concentration change after applying thapsigargin for 2 minutes in the Ca²⁺ free HEPES buffer; None (above), Caffeine (middle) or SKF96365(below),

FIGS. 9b and 9c show cyclopiazonic acid- or thapsigargin-induced increase in intracellular Ca²⁺ concentration ([Ca²⁺]ᵢ) in the Fura-2 loaded U178MG cells, without (control) or with caffeine treatment, and

FIG. 9d shows % of control by cyclopiazonic acid and thapsigargin in the presence of caffeine.

FIGS. 10a through 10c show inhibiting activity of caffeine analogs against Ca²⁺ release in brain tumor cells, wherein

FIGS. 10a and 10b show representative traces of blocks against TFLLR induced Ca²⁺ release, by Caffeine (a) and Theophylline (b), respectively, and

FIG. 10c shows % block by caffeine analogs.

### DETAILED DESCRIPTION OF THE PREFERRED EMBODIMENTS

A more complete appreciation of the invention, and many of the attendant advantages thereof, will be readily apparent as the same becomes better understood by reference to the following detailed description.

The present inventors found that caffeine and its analogs display inhibitory activity against proliferation, invasion and migration of brain tumor, in particular, glioma including blastoma (GBM), even if they used as a sole therapeutically active ingredient, thereby having an effective therapeutic activity against brain tumor, to complete the present invention.

Therefore, one embodiment of the present invention provides a composition for preventing and/or treating a brain tumor containing caffeine, its analog, and/or their pharmacologically acceptable salt. Another embodiment of the present invention provides functional food for preventing and improving a brain tumor, containing caffeine, its analog, and/or their pharmacologically acceptable salt. Another embodiment provides a method for prevention and/or treatment of a brain tumor, including administering a therapeutically effective amount of caffeine, an analog thereof, or a pharmacologically acceptable salt thereof, to a patient in need of the prevention and/or treatment of a brain tumor. The caffeine, an analog thereof, or a pharmacologically acceptable salt thereof may be exhibit the preventing and/or treating activity, even when it is used as a sole active ingredient with no other drug for treating a brain tumor, as shown in Example 5.

Caffeine is a kind of purine bases found in higher plants and has a xanthine structure with three methyl (CH₃) groups, as represented by the following chemical formula I (C₈H₁₀O₂N₄).

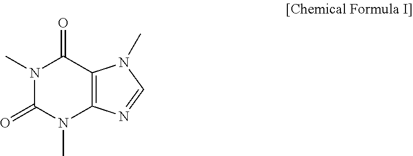

Caffeine is a white soft crystalline substance having properties as an excitatory component and exists in coffee beans at the amount of about 1 to 5%, in African kola nut at the amount of about 3%, in Paraguayan mate tea at the amount of about 1 to 2%, in Brazilian guarana berries at the amount of about 3 to 5%.

Caffeine may be isolated from green tea leaves by exuding with hot-water and removing substances like tannins. Alternatively it may be chemically synthesized from dimethylurea and malonic acid as starting materials. In plants, it is synthesized from such substances as glycine, formic acid, and carbon dioxide, in a similar manner to synthesis of other purine bases. Three methyl groups present in caffeine is originated from methionine. The importance of caffeine is in its pharmacological activities. It mainly displays activities as a CNS (central nervous s system) stimulant, a respiratory system stimulant, cardiotonic agent and diuretic agent. When applied in small amounts, caffeine is effective for fatigue recovery and relief of migraine and heart diseases. However, caffeine's therapeutic activity for brain tumor, specifically glioma, is firstly revealed by the present invention.

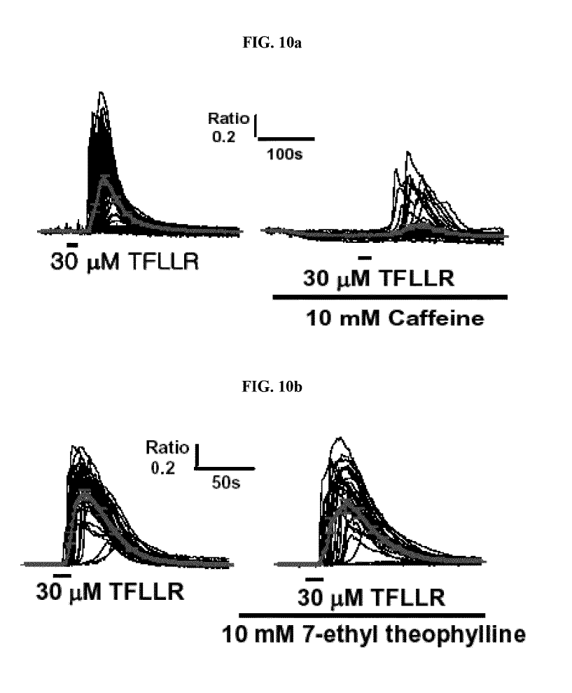

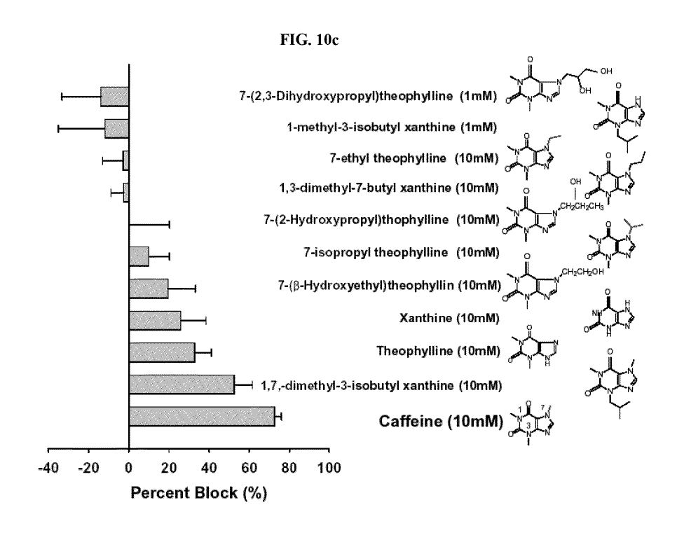

Caffeine analogs showing a therapeutic activity against a brain tumor may include 7-isopropyl-theophylline, 7(β-hydroxyethyl)theophylline, xanthine, theophylline, 1,7-dimethyl-3-isobutylxanthine, and the like. Such inhibitory activity of the caffeine analogs against IP₃R3 is as shown in FIG. 10.

The brain tumors that can be treated by the compositions according to the embodiment of the present invention may include those which originate in the brain itself and tissue, and those which have metastasized from other area of the body to brain, for instance one or more selected from the group consisting of glioma such as glioblastoma, astrocytoma and the like, meningioma, pituitary adenoma, schwannoma, craniopharyngioma, congenital brain tumor, and the like. Effects of prevention, treatment, improvement and/or alleviation by the compositions are particularly excellent when applied for glioblastoma (GBM). The compositions may be applied to mammals, preferably humans.

With regard to the amounts of Caffeine, analogs, and pharmaceutically acceptable salts to be contained in the compositions as an active ingredient, a desired amount may be in the range of 0.3 to 30 mM, preferably, 5 to 20 mM, and more preferably, 2.5 to 12 mM. Due to making the amount of the active ingredient in the compositions within the above range, the compositions can display sufficient efficacy, without cytotoxity caused by high concentration. In addition, daily doses of the compositions may be adjusted according to symptoms and intensities of the disease, and patient's conditions. It is preferred that a daily dose is determined within the range of 1 to 5 mg/kg (body weight), and the dose amount may be administered once or several times a day.

For the compositions according to the present inventions, caffeine, its analogs, and its pharmaceutically acceptable salts may be included in the compositions alone or together with other pharmaceutically acceptable medicine(s), carrier(s), or excipient(s). An appropriate range for the amount of caffeine, its analogs or their salt to be included in the compositions may be easily determined by those skilled in the relevant art according to the purposes of using the compositions. Types of carriers or excipients may be chosen depending on the formulated from of the compositions, and for example, conventionally used kinds of diluents, fillers, volume extenders, wetting agents, disintegrators, and/or surfactants are applicable for the compositions. Examples of typical diluents or excipients may include, but are not limited to, water, dextrin, calcium carbonade, lactose, propylene glycol, liquid paraffin, talc, isomerized sugar, sodium metabisulfite, methyl paraffin, propyl paraben, magnesium stearate, lactose, saline, colors and flavors.

The compositions may be orally or non-orally administered, with various types of formulations depending on desired manners of uses. Examples of formulations may include, but are not limited to, plasters, granules, lotions, powders, syrups, liquids and solutions, aerosols, ointments, fluidextracts, emulsions, suspensions, infusions, tables, injections, capsules and pills.

In addition, an embodiment of the present invention provides functional food for preventing and/or improving a brain tumor comprising caffeine, its analog, or their pharmaceutically acceptable salt. There is no specific limitation on the contents of caffeine, its analogs, or their pharmaceutically acceptable salt in said functional food, and such amount may be adjusted according to desired purposes or features of finished food products: for instance, a ratio of the active ingredient to the whole product weight may be determined within the range of 0.00001 to 99.9 weight %, preferably, 0.001 to 50 weight %. For the present invention, such functional food collectively refers to all types of food, health food supplements, and food additives. There is no limitation on the kinds of said food, health food supplements, and food additives. For instance, said foods can include: foods for special dietary uses (formulated milk, baby/infant formula, etc.), processed meat products, fish products, tofu, curd, noodles (ramen and other types of noodles), bread, functional food, seasoning food (soy bean sauce, soy bean paste, red pepper paste, mix paste, etc.), sauces, cookies and snacks, dairy products (fermented milk, cheese, etc.), otherwise processed food, kimchi, pickled food (sliced radish or cucumber seasoned with soy), drinks (fruit juice, vegetable juice, soy milk, fermented beverages, etc.); and can be one prepared by a commonly available method.

Below provided is a more detailed description of the present invention.

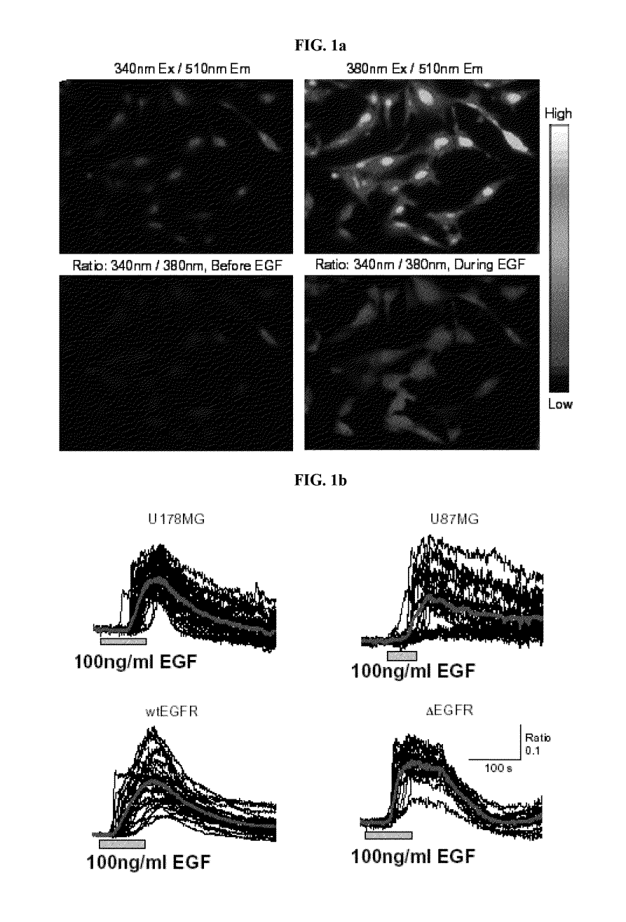

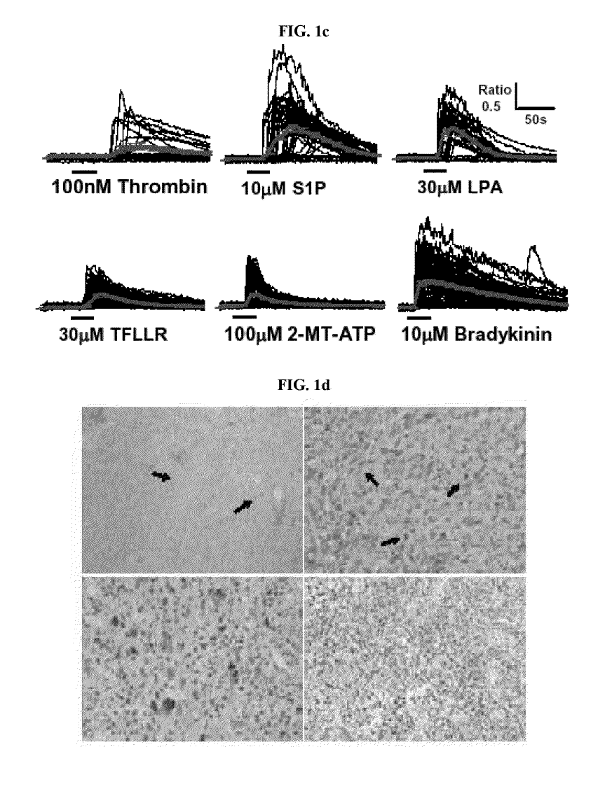

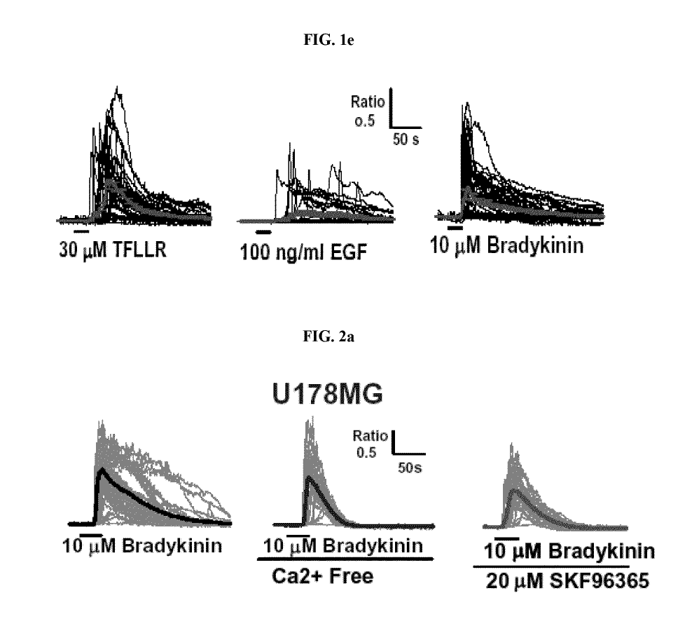

Glioblastoma Multiforme (GBM), the most malignant and invasive brain tumor, has a very poor prognosis, with a median survival of only one year after diagnosis. Complete surgical removal of GBM is very unlikely due to vagueness of boundary between brain and tumor. This difficulty basically results from the insidious propensity of these cells to migrate and invade into neighboring regions of the brain. Highly invasive GBM cells diffusely infiltrate the normal brain through the active killing of neurons, thereby securing their space. Various signaling molecules activate these GBM cells and affect their proliferation, motility, and invasiveness. Those signaling molecules include various growth factors such as epidermal growth factor (EGF), platelet-derived growth factor (PDGF), and the like, and other G-protein coupled receptor (GPCR) agonists such as ATP, bradykinin, lysophosphatic acid (LPA), sphingosine 1-phosphate (S1P), thrombin, plasmin, and the like. The signaling molecules then activate the surface receptors on their counterpart. Such surface receptors may be EGFR, PAR1, B2, P2Y, LPA receptor, S1P receptor, and etc., whose activation leads to activation of downstream effectors and, more importantly, induces an increase in intracellular Ca²⁺ concentration ([Ca²⁺]ᵢ)(FIG. 1).

FIG. 1 shows Ca²⁺ responses by various agonists for G-protein coupled receptor (GPCR) and receptor tyrosine kinase (RTK) agonists. FIG. 1a shows representative pseudo color fluorescence intensity images (380 nm or 340 nm excitation, 510 nm emission, top) and ratio images (below), of Fura-2 AM (5 μM) loaded glioblastoma cells before and after EGF stimulation. The bars on the right side indicate the degree of pseudo colors for fluorescence intensity; decreasing intensity goes toward the black side while increasing intensity goes toward the yellow side. FIG. 1b shows traces from Ca²⁺ imaging recordings performed for four glioblastoma cell lines. Each trace represents changes in Fura-2 AM intensity ratio in one cell (n=36 to 83 per cell line). The red line indicates average responses. Gray bars show duration of EGF application.

FIG. 1c shows changes in Fura-2 AM intensity ratio caused by various agonists on the Fura-2 loaded U178MG cells. FIG. 1d is a low power view of the GBM cells: the upper left side is a X200 image showing that cellular glial tumor has two foci of psudopalisading necrosis (arrows, H&E, X200); the upper right side is a X400 image showing frequent mitotic cells (arrows, H&E, X400); the left down side shows mutinulcleated pleomorphic nuclei. (H&E, X200); and the right down side shows that most of the tumor cells are immunoreactive to glial fibrillary acidic protein. (GFAP immunostaining, X200). FIG. 1e shows that agonists for GPCR and RTK induced intracellular Ca²⁺ increase in human glioblastoma cells.

Cancer cell migration depends mainly on actin polymerization and intracellular organization, where the actin polymerization and intracellular organization are influenced by various actin binding proteins. Regulation of actin binding protein activity is mediated by second messengers such as phosphoinositides and calcium. Therefore, the precise mechanism of Ca²⁺ increase in GBM cells may be considered as an important factor for controlling proliferation, motility, and invasiveness of the GBM cells. However, up to date, only limited amount of research has been conducted in regards to Ca²⁺ signaling in the GBM cells.

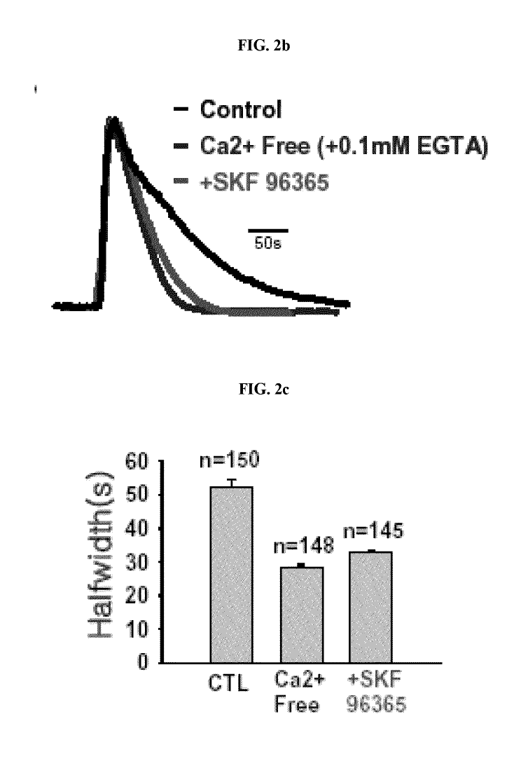

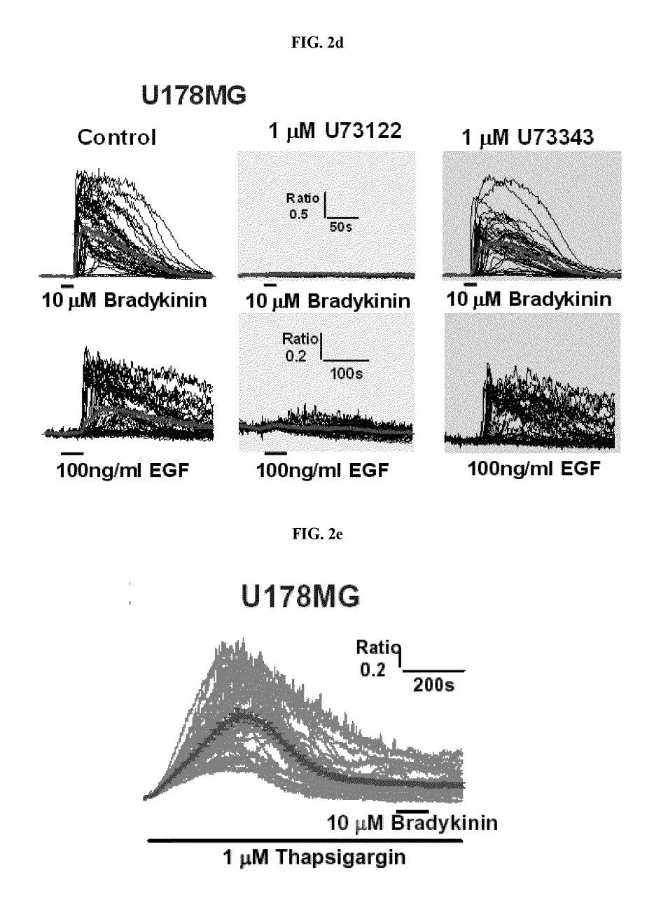

By performing Ca²⁺ imaging experiments for Fura2-AM loaded cultured GBM cell lines and acutely dissociated GBM cells prepared from surgically removed tissue, it was found that an increase in [Ca²⁺]ᵢ in these cells was contributable in part to a release of Ca²⁺ from intracellular release pools and in part to a Ca²⁺ entry through the store-operated channels (FIGS. 2a, 2b and 2c). The release of Ca²⁺ from intracellular stores was completely inhibited by U73122—a specific inhibitor of phospholipase C (PLC), which produces IP₃ by metabolism of phophoinositol-4,5-bisphosphate (PIP2)—in response to an activation of GPCRs and receptor tyrosine kinases (RTKs) (FIG. 1c).

FIG. 2 shows the results relating to Ca²⁺ signaling, where FIG. 2a shows that Fura-2 loaded U178MG cells were stimulated with bradykinin in 2 mM Ca²⁺ HEPES buffer, Ca²⁺-free HEPES buffer, and in the presence of SKF 96365, respectively. FIGS. 2b and 2c shows decay kinetics of each representative average trace and half width(s). Error bars represent SEM. FIG. 2d shows that pre-treatment of U73122 blocked bradykinin- or EGF-induced Ca²⁺ release rescued by pre-treatment of U73343. FIG. 2e shows the results of GPCR agonist application after depletion of Ca²⁺ by thapsigargin in endoplasmic reticulum. FIG. 2f shows decay kinetics of IP3R mediated Ca²⁺ release by bradykinin on U178MG cells in the presence of ryanodine receptor antagonist.

The Ca²⁺ entry through the store-operated channels appears to be tightly coupled to the release event, since store depletion by thapsigargin completely inhibited subsequent induction increase in [Ca²⁺]ᵢ by bradykinin (FIG. 2e). From these results we concluded that GBM cells express various surface receptors that are coupled to the common phosphoinositide pathway leading to Ca²⁺ release from intracellular stores and subsequent Ca²⁺ influx through store-operated channels. It is hypothesized that these molecules existing in the Ca²⁺ release pathway serve as a potential molecular target in controlling migration and invasion of GBM.

There exist two known channels that are responsible for release of Ca²⁺ from intracellular stores; IP₃Rs (inositol-1,4,5-triphospate receptor) and RyRs (ryanodine receptor). Caffeine has been classically known to induce release of Ca²⁺ from intracellular stores by opening RyRs, especially in muscle cells and cardiac myocytes. Thus, the present inventors tested caffeine along with other agents that enhance or disturb the Ca²⁺ release machinery in various assays conducted for GBM motility, invasion, and proliferation.

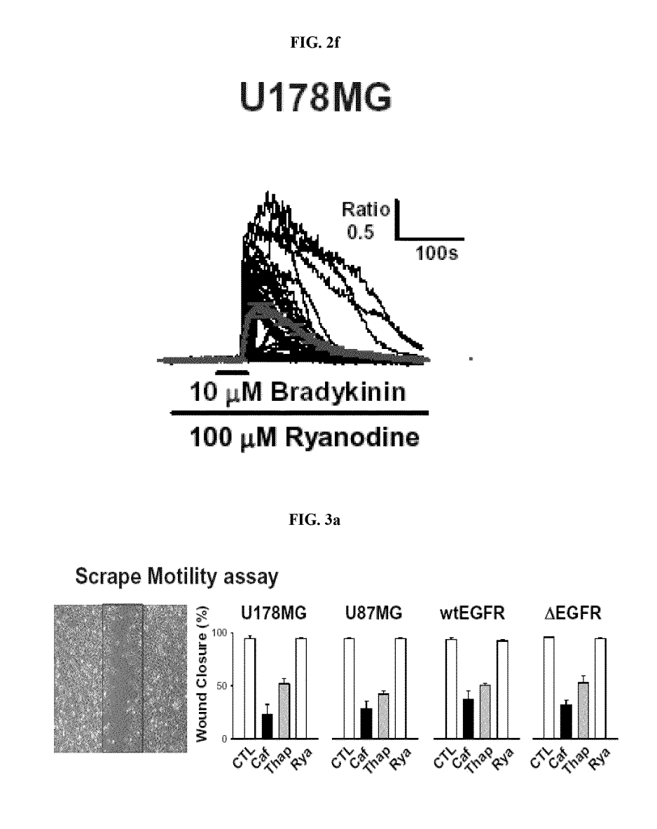

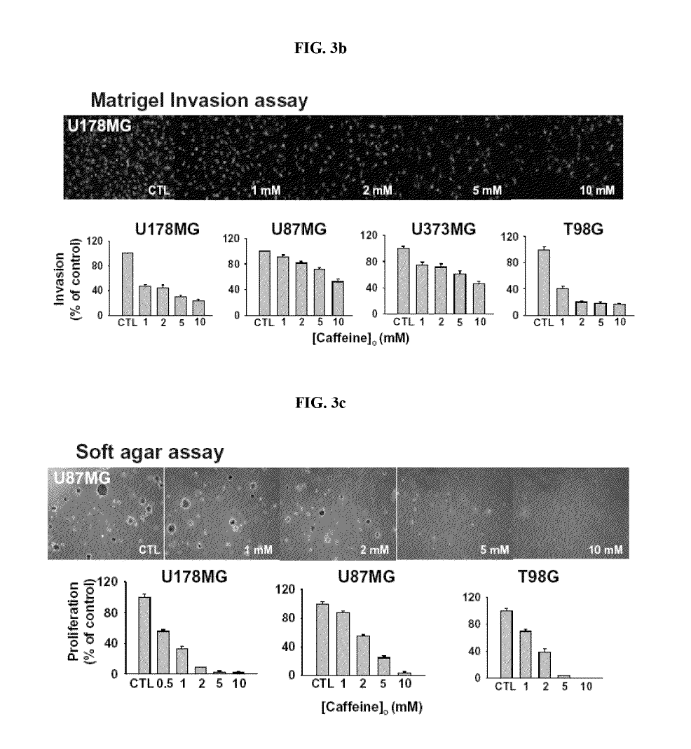

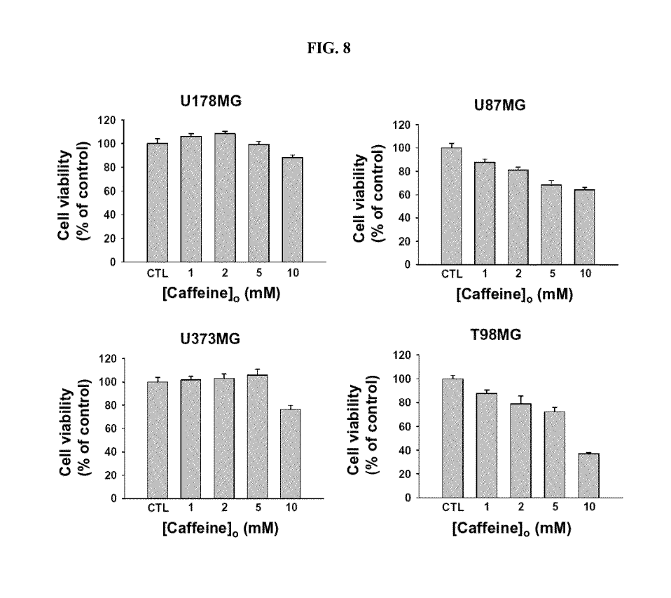

In contrast to the inventors' expectations, it was found that 10 mM caffeine significantly inhibited the motility, invasion, and proliferation of various GBM cell lines, U178MG, U87MG, U373MG, and T98G cells (FIGS. 3a, 3b, 3c), while minimally affecting the cell viability (FIG. 8). This paradoxical effect of caffeine was mimicked by various agents such as 1 μM thapsigargin, 10 μM 2-APB, and 20 μM CPA, 50 μM BAPTA-AM, and the like, which are known to disturb the release of Ca²⁺ from intracellular stores, but not by 10 μM ryanodine, an agonist of RyRs at this concentration (FIG. 3a).

The above fact suggests that caffeine's mode of action is involved with inhibited Ca²⁺ release from intracellular stores and that the inhibitory action is selectively targeting to IP₃Rs, not RyRs. The experiment in the present invention revealed that inhibitory action exhibited by caffeine and its analogs is specific to IP₃R3 in the brain tumor, in particular, glioblastoma, thereby inhibiting IP₃R 3-mediated Ca²⁺ increase. By such activity, caffeine and its analogs are capable of inhibiting proliferation, migration, and invasions signalized by Ca²⁺, thereby exhibiting excellent therapeutic activity against glioblastoma.

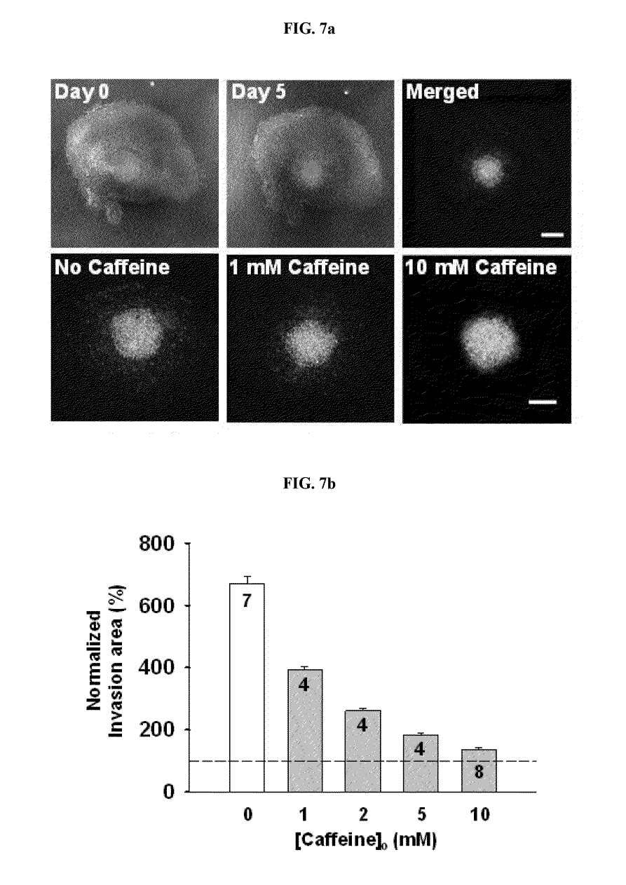

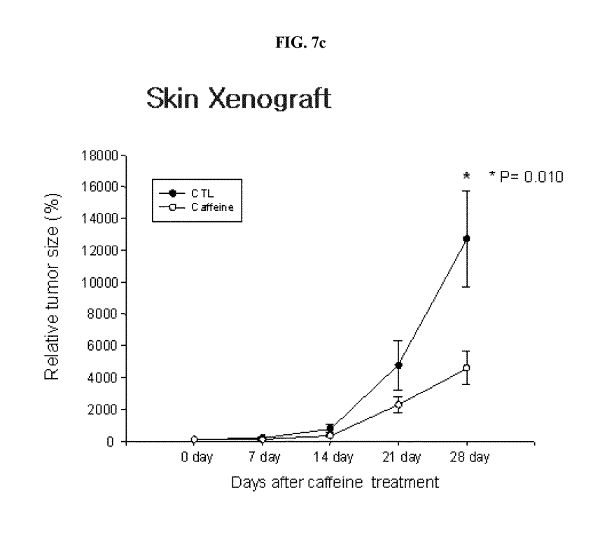

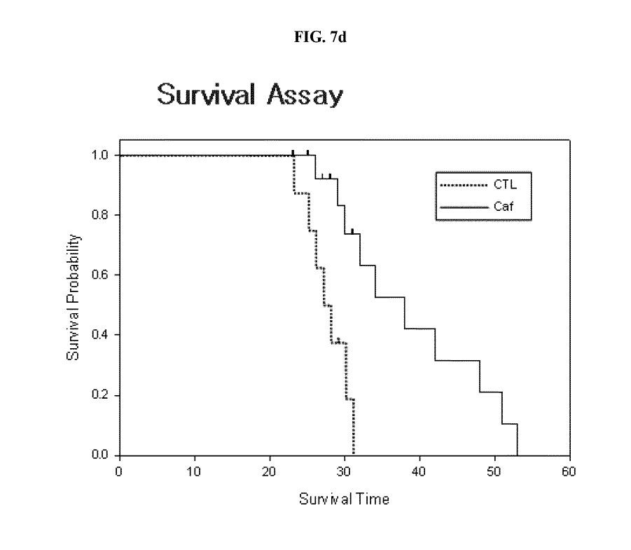

To translate the results from the in vitro experiments to more systemic level, a test was conducted to examine the effect of caffeine on acute slice and in vivo animal model in which local microenvironments could compromise the effect of caffeine. From the result, it is found that migration, invasion, and proliferation of GBM cells are significantly lowered in the animal model with the treatment caffeine (see FIGS. 7b and 7c).

The present invention provides a detailed molecular mechanism for caffeine action on migration and invasion of GBM that is known as the most fatal kind of brain tumors. The beneficial effect of caffeine is expected to be applied to the treatment of other fatal diseases with a similar mechanism to brain tumor but having no treating means.

### Example 1

### Example 1

### Preparation of Glioblastoma Cells

Glioblastoma cell lines were maintained in Dulbecco's modified Eagle's medium (DMEM; Gibco) supplemented with 10% fetal bovine serum (FBS), 1% L-glutamine, 1% sodium pyruvate, penicillin (50 units/mL), and streptomycin (50 units/mL). Human glioblastoma were maintained in DMEM supplemented with 20% FBS and 1% L-glutamine, 1% sodium pyruvate, penicillin (50 units/mL), and streptomycin (50 units/mL).

### Example 2

### Assays to Test Caffeine's Inhibiting Effect on Mobility, Invasion and Proliferation of GBM Cells

Assays were conducted to test caffeine's inhibiting activity against mobility, invation and proliferation of various GBM cell lines.

2.1. Scrape Motility Assay to Test Caffeine's Inhibiting Effect on Mobility

To first test caffeine's effect on mobility, U178MG, U87MG, wtEGFR, and ΔEGRF cells were used as glioma cell lines. The cell lines were obtained from Emory Uni (U178MG) and ATCC (U87MG, T98G, and M59K). All cell lines were grown as monolayers in 12-well culture plates in serum-containing media (Emory Uni). Scrapes were made with a 10 μL pipet tip, drug (10 mM of caffeine, 1 μM of thapsigargin, or 10 μM ryanodin) was added, and the plates were returned to the incubator, allowing the cells incubated (n=3 to 4 per each cell line, 37). To prevent proliferation, fluorodeoxyuridine (FdU/U; Sigma) was added. After incubation for 24 hours, the cells were fixed in 4% paraformaldehyde. The area of repopulation of three 10× fields within the scrape areas were determined and the mean percentage of scrape area to wound closure was determined. The cell line without caffeine treatment was used as a control.

The obtained result is shown in FIG. 3a. The boxed area in the left side indicates approximates borders of the scrapes. Data shown in the graph on the right side indicates percentage of wound closure by cell migration. As observed from the above, caffeine induced depletion of intracellular Ca²⁺ stores—in a similar manner of thapsigargin treatment that depletes intracellular Ca²⁺—, thereby highly effectively inhibiting the migration of cells into the wound area. However, treatment with ryanodine, an agonist for RyRs (ryanodine receptors) that is one of intracellular Ca²⁺ concentration related receptors, did not show any cell migration as such. Error bars are mean±SEM. **p <0.01, ANOVA with Newman-Keuls post hoc.

2.2. Matrigel Invasion Assay to Test Caffeine's Inhibiting Effect on Invasion

To test caffeine's inhibiting effect on invasion, U178MG, U87MG, U373MG, and T98G cells were used as glioma cells lines. The cell lines were obtained from ATCC. 1, 2, 5, and 10 mM of caffeine were added to the cell lines, respectively (n=4). Cell invasion was assayed using transwell inserts (Corning, N.Y., USA) containing 8 uM pore size in 24-well culture plates. For invasion assay, inserts were coated with 2 mg/ml basement membrane Matrigel (BD Bioscience, Bedford, Mass., USA). 1×10⁵ cells in serum-free medium (FBS, DMEM, from GIBCO, Invitrogen, USA) were plated onto the upper side of insert and complete medium was placed in the lower chamber to act as a chemoattractant. After 24 h of incubation at 37, the cells on the upper side of insert were removed by wiping with a cotton swab and cells migrated to the lower side of membrane were stained with DAPI (Molecular Probes, Invitrogen, USA) and randomly photographed with microscopy at X40 magnification. The mean number of untreated cells was considered as 100% invasion.

FIG. 3b shows invasion of the cells as obtained from the above; top is representative pictures of cells that invaded through Matrigel at various concentrations of caffeine, and bottom is a graph showing percentage of invaded cells to the control. The invaded cells were counted at ×20 magnification under microscope. The assay was duplicated and five fields randomly selected and counted for each assay. As can be seen from the results, caffeine reduced the invasion rate of glioblastoma cells, in a dose dependent manner (reduction increasing proportionally to amount of caffeine treated) after the treatment for 24 hours.

2.3. Soft Agar Assay to Test Caffeine's Inhibiting Effect on Colony Formation

To test caffeine's inhibiting effect on proliferation, soft agar assay was conducted. Cells (1×10⁴) were seeded into 6-well plates in a soft agar (0.3%, Difco) overlaying a 0.6% base agar. The solidified cell layer was covered with medium containing 0.5, 1, 2, 5, or 10 mM of caffeine which was replaced every 4 days. Cells were incubated at 37 for 14 to 17 days to allow colonies to develop. Afterward colonies were stained with 0.05% cresyl violet and photographed. The group without caffeine treatment was used as a control. Each assay was done in triplicate (n=3)

FIG. 3 shows the results as obtained from the above; top is a photograph showing colonies formed at various concentrations of caffeine, and bottom is a graph indicating the percentage of colonies numbers to the control. As shown in FIG. 3, caffeine reduced the anchorage-independent growth of glioma in vitro, in a dose dependent manner.

### Example 3

### Caffein's Block Against Intracellular Ca²⁺ Release

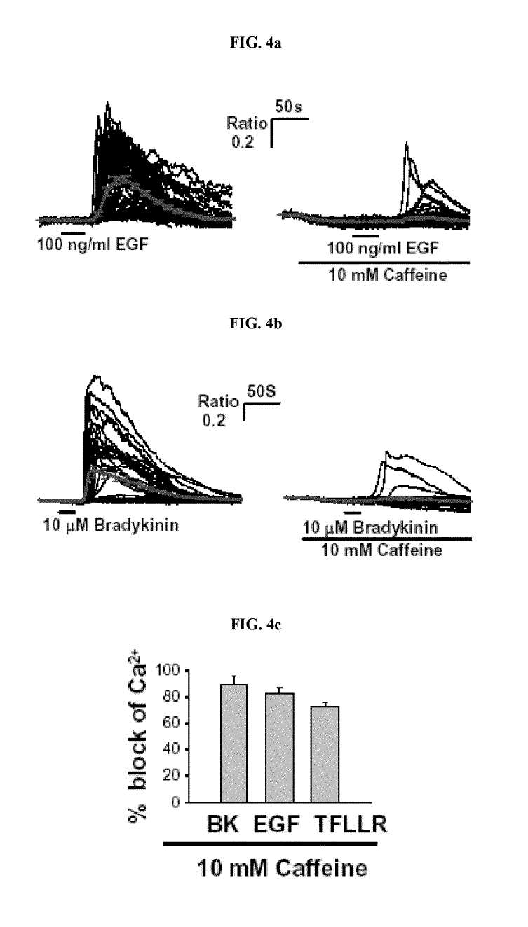

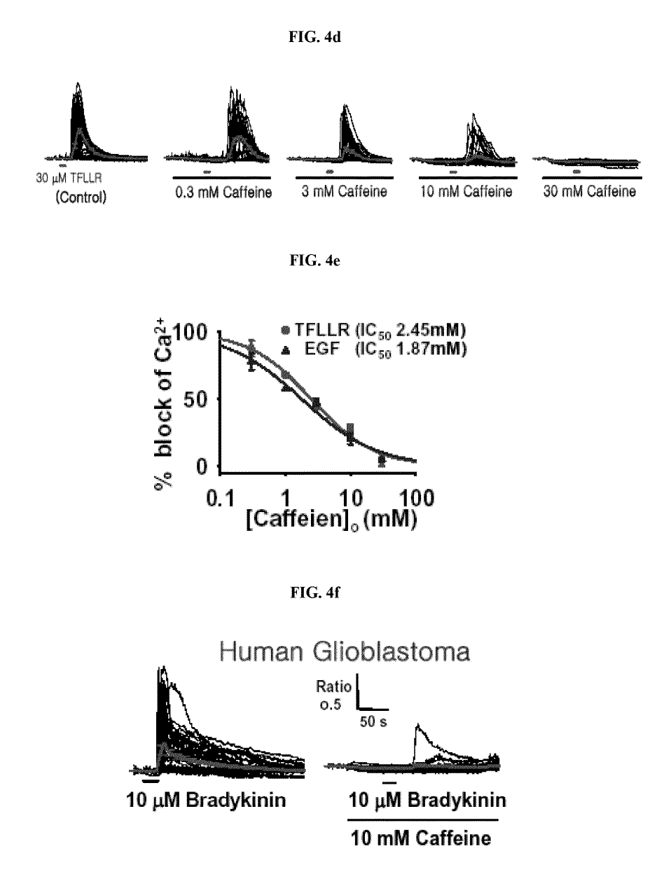

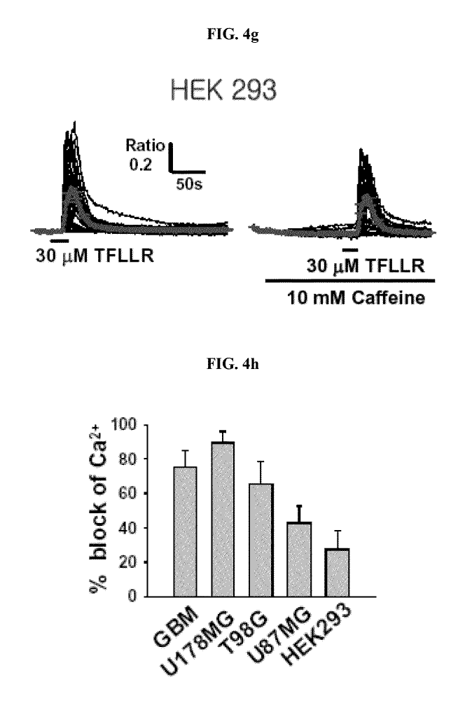

U178MG cells were treated with 10 mM caffeine. 100 seconds after the treatment, the cells were divided into two separate groups, each being stimulated with GPCR (G-protein coupled receptors) agonists, i.e., 100 ng/ml EGF or 10 μM bradykinin, respectively. Inward current was measured, to test the intracellular Ca²⁺ release block by caffeine in the U178MG cells stimulated with EGF or bradykinin. The measurement of Ca²⁺ concentration through the measurement of inward current was conducted as described in ‘Justin Lee, et al., The Journal of Physiology, Astrocytic control of synaptic NMDA receptor, 2007’, which is hereby incorporated by reference for all purposes as if fully set forth herein. The group without caffeine treatment was used as a control. As indicated in FIGS. 4a and 4b, no significant increase of [Ca²⁺]i was in the cells treated with caffeine, although treated with the agonists.

100 seconds after treating U178MG cells with 10 mM caffeine, U178MG were stimulated with various agonists (10 μM bradykinin(BK), 100 ng/ml EGF, 30 μM TFLLR). FIG. 4c shows % block by caffeine against agonist-induced Ca²⁺ release on U178MG. Error bars are mean±SEM. As known from FIG. 4c, caffeine displays blocking effect against intracellular Ca²⁺ release, for various Ca²⁺ release agonists.

U178MG cells were treated with 0.3 mM, 3 mM, 10 mM, and 30 mM of caffeine, respectively. After 100 seconds, the cells were stimulated with 30 μM TFLLR. FIG. 4d shows the blocking effect by caffeine against TFLLR-induced Ca²⁺ release on U178MG. As known from FIG. 4d, caffeine inhibits the increase of TFLLR-induced intracellular Ca²⁺ concentration, in a caffeine concentration dependent manner.

FIG. 4e shows dose response curve of Ca²⁺ release evoked by TFLLR (30 μM) and EGF (100 ng/ml), depending on varying concentrations of caffeine. Determined IC₅₀ values were 2.45 mM for TFLLR and 1.87 mM for EGF.

FIGS. 4f and 4g show behaviors of intracellular Ca²⁺ release in human glioblastoma cell line (SH-SY5Y, ATCC) and HEK293T cell line (ATCC), where the cells were treated with 10 mM of caffeine and then stimulated with 10 μM bradykinin (for human glioblastoma cells) or 30 μM TFLLR (for HEK293). As can be known from FIGS. 4f and 4g, intracellular Ca²⁺ release was blocked by caffeine in both cell lines.

FIG. 4h shows % block by caffeine (10 mM) against GPCR-induced intracellular Ca²⁺ release in GBM, U178MG, T98G, U87MG and HEK293. The cell lines were obtained from Department of Neurosurgery, Seoul National University College of Medicine (GBM), Emory Uni. (T178G) and ATCC (T98G, U87MG, and HEK293), respectively. It was found that intracellular Ca²⁺ release is inhibited by caffeine in most of the cells. Error bars are mean±SEM.

### Example 4

### Tests to Evaluate Selective Block Against IP₃R Subtype 3 (IP₃R3)

4.1. Determination of mRNA Expression

Measurements were conducted for mRNA expressions of IP₃Rs and glyceraldehyde-3-phosphate dehydrogenase (GAPDH) in human glioma cell lines (U87MG, U178MG, U373MG, T98G, and M059K), human glioblastoma cell line (SH-SY5Y), and HEK293T cell line. mRMA expressions were measured by RT-PCR (Reverse transcription polymerase chain reaction). Total RNAs were separated from the above prepared samples with TRIZOL® reagent (Invitrogen, Carlsbad, Calif.), and 1 ug of the separated RNA was amplified. Each cycle consisted of 30 seconds at 94 for denaturation, 30 seconds at 55 for annealing, and 60 seconds at 72 for extension. The actual sequences of specific primers were as follows.

(SEQ ID NO: 1)

| | |
|---|---|
| IP₃R1 sense: | 5′-CTCTGATCGTTTACCTG-3′, |

(SEQ ID NO: 2)

| | |
|---|---|
| ITPR1 antisense: | 5′-TCTTCTGCTTCTCACTCCTC-3′; |

(SEQ ID NO: 3)

| | |
|---|---|
| IP₃R2 sense: | 5′-AGAAGGAGTTTGGAGAGGAC-3′, |

(SEQ ID NO: 4)

| | |
|---|---|
| IP₃R2 antisense: | 5′-TCACCACCTTTCACTTGACT-3′; |

(SEQ ID NO: 5)

| | |
|---|---|
| IP₃R3 sense: | 5′-CTGTTCAACGTCATCAAGAG-3′, |

(SEQ ID NO: 6)

| | |
|---|---|
| IP₃R3 antisense: | 5′-CATCAACAGAGTGTCACAGG-3′; |

(SEQ ID NO: 7)

| | |
|---|---|
| GAPDH sense: | 5′-AGCTGAACGGGAAGCTCACT-3′, |

(SEQ ID NO: 8)

| | |
|---|---|
| GAPDH antisense: | 5′-TGCTGTAGCCAAATTCGTTG-3′. |

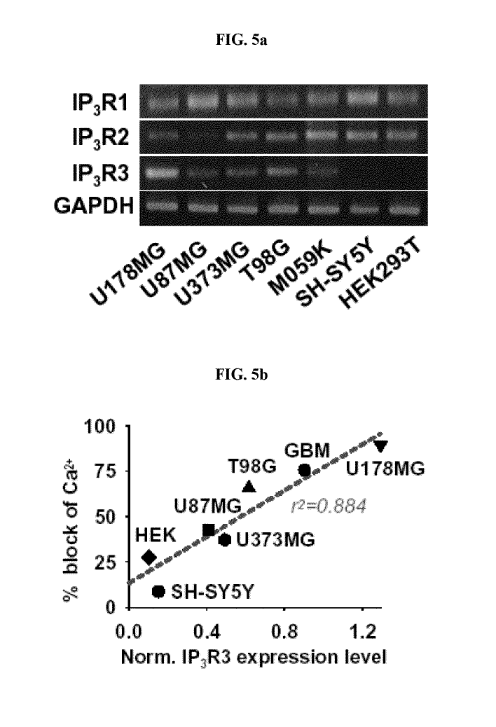

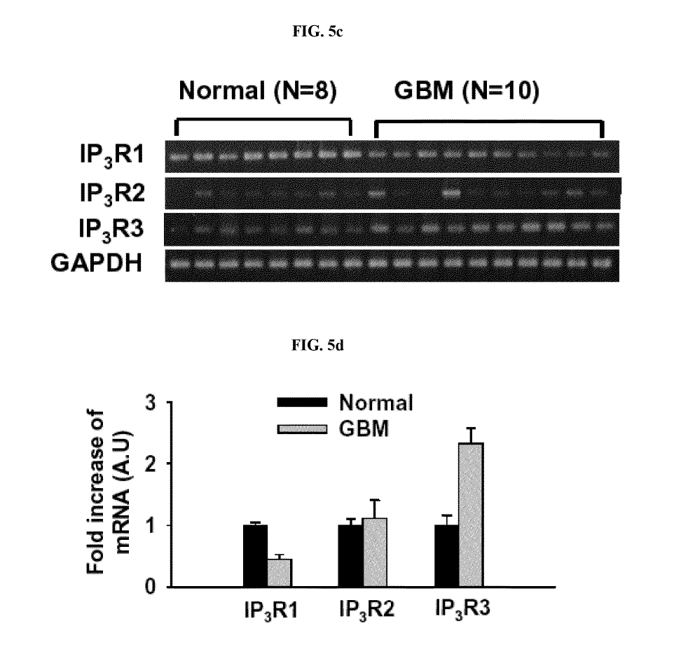

FIG. 5a is the result for mRNA expression obtained by electrophoresis of the above obtained PCR products. The degrees of three subtypes of IP3R mRNA expressions were different for respective cell lines.

FIG. 5b shows correlation between the degree of IP₃R3 expression and Ca²⁺ block by caffeine in respective cell lines. Ca²⁺ level was determined according to methods as described in Examples 2 and 3. As indicated in FIG. 5b, a statistically significant correlation was found between the expression level of IP₃R3 and Ca²⁺ block. This suggests that caffein's inhibitory activity is linked with IP₃R3.

FIG. 5c shows a comparison between IP₃R subtype mRNA expressions in normal human brain cells (n=8, Department of Neurosurgery, Seoul National University College of Medicine) and human glioblastoma cells (n=10), as performed on electrophoresis. FIG. 5d shows densitomeric histograms of IP₃R mRNA expression in the above human samples. As can be known from FIGS. 5c and 5d, the expression of IP₃R3 was considerably high in the glioblastoma cells, compared to other IP₃R subtypes.

4.2. Caffeine's Activity Specific to IP₃R3

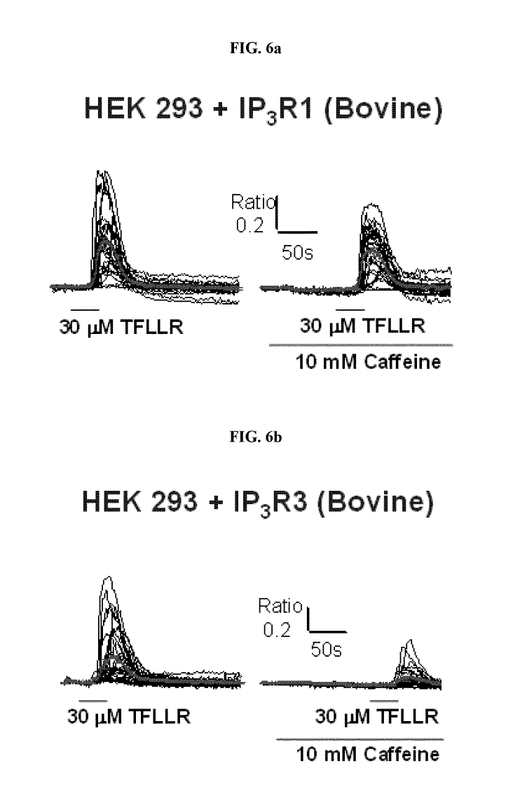

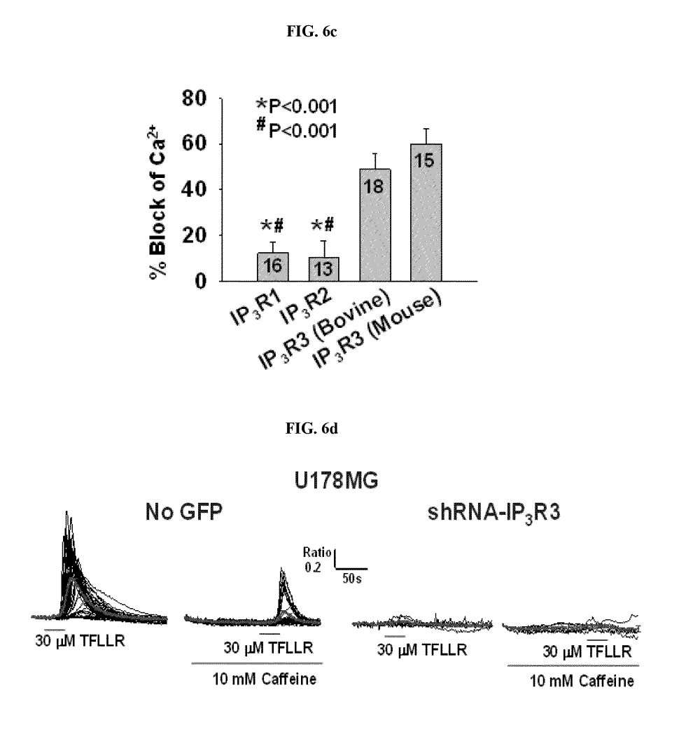

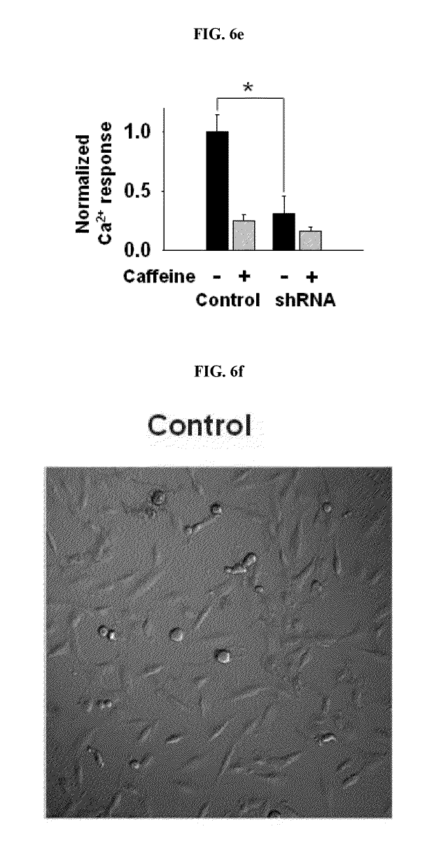

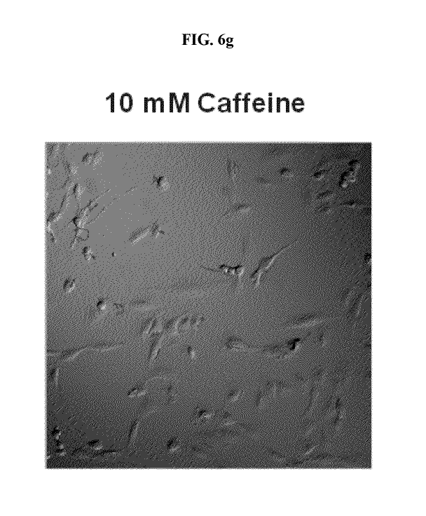

HEK293T cells were transfected with IP₃R1 (Bovine) and IP₃R3 (Bovine), respectively (the HEK293T cells were obtained from ATCC, which is co-transfected with GFP and IP₃R gene, and Ca²⁺ imaging experiments were performed only for GFP-transfected cells, where the Ca²⁺ imaging was conducted 2 days after transfection). The cells were then treated with 10 mM caffeine. For the caffeine-treated cells, the degree of block against TFLLR-induced Ca²⁺ release (with 30 μM TFLLR treatment) by caffeine was assessed. The results are as shown in FIGS. 6a and 6b. In FIG. 6a, when IP₃R1 was expressed, block by caffeine against TFLLR-induced Ca²⁺ release was not noticeable. However, as seen from FIG. 6b, when IP₃R3 was expressed, considerable block by caffeine against TFLLR-induced Ca²⁺ release was observed.

HEK293T cells were transfected with IP₃R1 (Bovine), IP₃R3 (Bovine), and IP₃R3 (Mouse), respectively (the HEK293T cells were obtained from ATCC, which is co-transfected with GFP and IP₃R gene, and Ca²⁺ imaging experiments were performed only for GFP-transfected cells, where the Ca²⁺ imaging was conducted 2 days after transfection). The cells were then treated with 10 mM caffeine. For the caffeine-treated cells, % block against TFLLR-induced Ca²⁺ release (with 30 μM TFLLR treatment) by caffeine was assessed. The result is as shown in FIG. 6c. As seen from FIG. 6c, block by caffeine against TFLLR-induced Ca²⁺ release was specific to IP₃R3, for the both genes originated from mouse as well as from bovine. Error bars are mean±SEM.

FIGS. 6d and 6e are Ca²⁺ imaging result for U178MG cells, which were transfected with GFP-attached shRNA for IP₃R3 via electroporation. Little Ca²⁺ release was observed in the shRNA-expressing cells, while normal release of Ca²⁺ was observed in the cells transfected with GFP only. This shows that caffeine addition significantly blocks Ca²⁺ release. From the experiment, therefore, it is now established that IP₃R3 plays a critical role in Ca²⁺ release in glioma cells and that caffeine displays inhibitory action against Ca²⁺ release specific to IP₃R3.

In addition, FIGS. 6f and 6g show live imaging results for cell migration of U178MG cells with and without caffeine treatment, where cell migration, which was observed through a microscopy (×200) with 10 minute intervals for 9 hours, was traced in red line. As seen in FIGS. 6f and 6g, migration of U178MG cells was significantly slowed down when treated with caffeine.

### Example 5

### Inhibition by Caffeine of Tumor Growth

It was tested whether caffeine reduces invasion of U178MG glioma cells in organotypic hippocampal slice cultures (OHSCs). Said OHSCs were prepared as descried in ‘Simoni A D and Yu L M, Preparation of organotypic hippocampal slice cultures: interface method. Nat. Protoc. 2006; 1(3):1439-45’. Some alteration was made to the organotypic glioma invasion (Eyüpoglu I Y, Hahnen E, Buslei R, Siebzehnrübl F A, Savaskan N E, Lüders M, Tränkle C, Wick W, Weller M, Fahlbusch R, Blümcke I. Suberoylanilide hydroxamic acid (SAHA) has potent anti-glioma properties in vitro, ex vivo and in vivo. J. Neurochem. 2005 May; 93(4):992-9). In short, Dil-strained U178MG cells (5000 cells/20 nl) were mounted on the 6 day aged-organotypic hippocampal slice cultures, in the presence of 0, 1, 2, 5, and 10 mM of caffeine. After 1 hour and 120 hours, behaviors of the glioma cells were observed using inverted confocal laser scanning microscope (Zeiss LSM5, Carl Zeiss, Germany). The result obtained is shown in FIG. 7a. FIG. 7a is a photo showing the U178MG cells placed on the 6 day aged-organotypic hippocampal slice cultures, where the photo shows the two images—respectively taken after 1 hour and after 120 hours—were interposed using Adobe Photoshop 7 software (scale bars: 500 μm).

In addition, invasion area of the DiI-stained cells was determined using Image J software (NIH, MD).

Invasion Area(%)=(area of DiI-stained cells after 120 hours/area of DiI-stained cells after 1 hour)×100.

The results of the calculation of invasion area are shown in FIG. 7b. Data is presented as mean±SEM (***p<0.001 by Students t-test; vs. control; +++p<0.001 by Student's t-test, vs. non-treated). As seen in FIG. 7b, it was found that invasion area was reduced with caffeine treatment, compared with no-caffeine treatment case and that the area reduction was made proportional to the concentration of caffeine treated.

In addition, a test using a xenograft model was conducted to examine caffeine's inhibitory effect on tumor growth, where U78MG cells (ATCC) were injected into the skin, and the progress of the tumor was examined. Five-week-old athymic mice (Balb/c nu/nu) were obtained from Central Lab. Animal Inc. (Japan). For the xenograft tumor growth assay, U87MG cells (3×10⁵ cells/150 ul PBS) were injected subcutaneously into the right flanks of the mice (n=5 to 10 mice per group), and the experiment was conducted in triplicate. At 7 days after injection, caffeine (Sigma, St. Louis, Mo.) was given through drinking water at the concentration of 1 mg/ml. The control animals were given distilled water. Tumor mass was estimated twice per week for 4 weeks, and tumor volumes were calculated by the formula:

volume=length×width²/2.

The effect of the caffeine was determined by the growth delay of the tumor cells.

As shown in FIG. 7a, the increase of tumor mass was significantly inhibited when treated with caffeine, compared with the control where caffeine was not treated. The FIG. 7b shows the growth of tumor mass by %, giving 100% to the mass of the day (day 0) when the caffeine treatment was initiated.

To translate the results from the in vitro experiments to more systemic level, the effect of caffeine was examined on acute slice and in vivo animal model in which local microenvironments could compromise the effect of caffeine. In acute mouse brain slices, 1 μl of DiI loaded U178MG cells were placed in striatum region and the radial progression of these cells to neighboring regions was examined. As indicated in FIG. 7c, it was found that the invasion of DiI loaded U178MG cells showed significantly lower invasion in the brain slices that were treated with 10 mM caffeine, compared to the control slices in which 10 mM 7-ethyl theophylline and no caffeine (0 mM) were treated.

To test the effect of caffeine on survival rate, orthotropic implantation model was built in which human U87MG was implanted. In order to prepare the orthotropic implantation model, pretreatment with caffeine solution (0.1% wt/vol) was first conducted for 1 week. Then U87MG cells (1×10⁴ cells/5 ul PBS) were implanted by intracranial injections in the left frontal lobe at coordinates 2 mm lateral from the bregma, 0.5 mm anterior, and 3.5 mm intraparenchymal. For the GBM animal model in which U87MG cells were injected to the brain of a nude mouse (5 wk-old, Balb/c nu/nu), survival rates was measured for mice supplied with 1 mg/ml caffeine containing drinking water and for mice not supplied so. The result is shown in FIG. 7d. Survival rate is a period between the time when the tumor was injected (day 0) and when the mouse died, which is indicated in graph in FIG. 7d. The control (CTL) is the group not treated with caffeine. As shown in FIG. 7d, mice supplied with caffeine show significantly increase the survival rate, compared to the control mice. This indicates that caffeine treatment in mouse model greatly reduces invasion and proliferation of GBM cells.

### Example 6

### Cytotoxicity Assays

Cell viability of each of U178MG, U87MG, U373MG, and T98MG cell lines depending on caffeine concentration was assessed by colorimetric MTT reduction assay. Cells were grown in a 96-well plate prior to caffeine treatment. After 24 h of treatment, 10 ul of MTT solution (2.5 mg/ml) was added to each well, and the cells were incubated for 4 hours at 37. Cells were solubilized with DMSO and quantified spectrophotometrically at 570 nM. Data were presented as the percentage of viability relative to control value.

The result obtained is shown in FIG. 8. Data in FIG. 8 is the survival rate relative to the control group (without caffeine treatment). As can be seen in FIG. 8, a decreased survival rate was found with 10 mM caffeine treatment in the T98MG cell line, survival rates were higher (70% or more) in other cell lines. This suggests that caffeine exhibits low level of cytotoxicity in a relatively high concentration.

### Example 7

### Correlation Between Caffeine Action and Ca²⁺ Concentration

In order to see whether caffeine action is dependent on store-operated channels or store depletion, caffeine actions in Ca²⁺- and Ca²⁺ free-baths were tested.

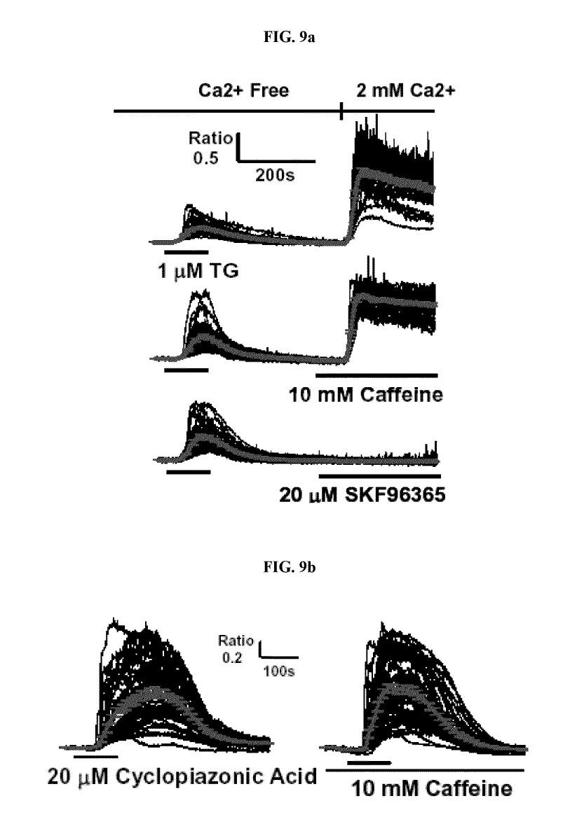

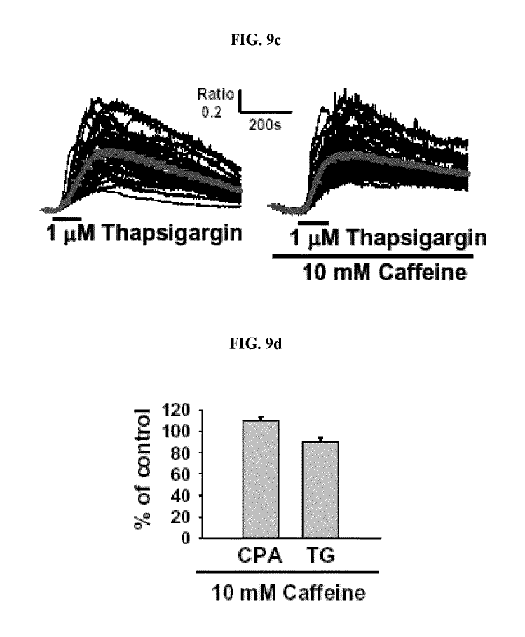

Firstly, 1 μM thapsigargin was applied for 2 min in Ca²⁺ free HEPES buffer. After depletion of Ca²⁺ from endoplasmic reticulum, extra solution was changed to 2 mM Ca²⁺ HEPES buffer. 10 mM caffeine or 20 μM SKF96365 (U178MG cell line, Emory Uni.) was applied 100 seconds before switching of extra solution. Resulting Ca²⁺ changes are indicated in FIG. 9a: None (above), Caffeine (middle) or SKF96365 (below).

FIGS. 9b and 9c show cyclopiazonic acid (20 μM)-induced or thapsigargin (1 μM)-induced increase in [Ca²⁺]i in the Fura-2 loaded U178MG cells, without (control) or with 10 mM caffeine treatment. FIG. 9d shows % of control by cyclopiazonic acid (20 μM) and thapsigargin (1 μM) in the presence of caffeine. Error bars are mean±SEM.

FIGS. 9a-9d demonstrate that Ca²⁺ block by caffeine occurs block of Ca²⁺ release through IP₃R. That is, Ca²⁺ block by caffeine is not due to Ca²⁺ depletion or block of Ca²⁺ flow through TRPC (transient receptor potential ion channels). Rather it occurs by block against IP₃R.

### Example 7

### Test of Action by Caffeine Analogs

In order to evaluate whether caffeine analogs as well as caffeine possess equivalent level of activity to caffeine, in other words, whether such analogs possess inhibiting activity against Ca²⁺ release in brain tumor cells and against proliferation, migration, and invasion by Ca²⁺ signaling, inhibiting activity of several caffeine analogs against Ca²⁺ release was tested.

First, U178MG cells (Emory Uni.) were treated with 10 mM caffeine and 10 mM 7-ethyl theophylline, respectively, and then, with 30 μM TFLLR. Behaviors of intracellular Ca²⁺ release were estimated and the results are shown in FIG. 10a (caffeine) and FIG. 10b (7-ethyl theophylline). As shown in the FIGS. 10a and 10b, TFLLR induced Ca²⁺ release was inhibited by caffeine treatment, but with no inhibition found by its analog, 7-ethyl theophylline. Therefore, it was found that not all caffeine analogs display blocking effect similar to caffeine.

To search substances having significant blocking effect among caffeine analogs, 10 representative caffeine analogs were examined on their blocking effects against Ca²⁺ release (% block). The results are shown in FIG. 10c. Error bars indicate SEM. As observed from FIG. 10c, other substance than Caffeine were found to have some degree of blocking effects against Ca²⁺ release. Such substance may include: iso-propyl theophylline, 7-(β-hydroxyethyl)theophylline, xanthine, theophylline, and 1,7-dimethyl-3-isobutyl xanthine. Among these substances, 7-(β-hydroxyethyl)theophylline, xanthine, theophylline and 1,7-dimethyl-3-isobutyl xanthine exhibit excellent inhibiting effect of 20% or more, and, in particular, 1,7-dimethyl-3-isobutyl xanthine exhibits very excellent inhibiting effect of 50% or more.

### Example 8

### Microarray Analysis of Genes for Ca²⁺ Signaling Pathway

The microarray analysis used in this example was conducted in the following manner.

8.1. Extraction of total RNAs

Total RNAs were isolated from human 10 normal brain tissue samples and 27 glioma samples using TRIZOL® reagent (Invitrogen, UK) according to the manufacturer's instructions, and purified by RNeasy mini kit (Qiagen, Valencia, Calif.).

8.2. Assessment of RNA Quantity, Integrity and Purity

Total RNA quantity and purity were assessed by measuring OD_{260/280} using a NanoDrop spectrophotometer (NanoDrop Technologies, Wilmington, Del., USA). RNA with an A260/280 ratio of >1.8 is considered acceptable for microarray experiment. RNA length distribution and integrity were assessed by capillary electrophoresis with fluorescence detection (Agilent Bioanalyzer 2100) using the Agilent Total RNA Nano chip assay (Agilent Technologies, Palo Alto, Calif.) for presence of 28S and 18S rRNA bands. Ideally, the intensity of the 28S band should be twice the intensity of the 18S.

8.3. Microarray Platform

Gene expression analysis was conducted using the Agilent Human 1A(V2) oligo microarry Kit (Agilent Technologies, Palo Alto, Calif.). The microarray was designed with four replicates of each probe distributed across the array, that is, 4×20K Multiplex slide format. Each of the four replicates contains more than about 20,000 of 60-mer—include control spot—Human genes and transcripts sequences.

8.4. RNA Label and Hybridization

Fluorescence-labeled cRNA probes for oligo microarray analysis were prepared by amplification of total RNA in the presence of aminoallyl-UTP using Amino allyl MessageAmp™ aRNA kit (Ambion Inc., Texas), followed by the coupling of Cy3 or Cy5 dyes—Incase the 1 color use Cy3 dye—(AmershamPharmacia, Uppsala, Sweden). Hybridizations were performed at 65 for 17 h in a rotating hybridization oven using the Agilent 60mer oligo microarray processing protocol. Slides were washed as indicated in this protocol and then scanned with a GenePix 4000B Array Scanner (Axon Instruments, Union City, Calif.).

8.4. Microarray Data Analysis

Scanned images were analyzed with GenePix Pro 6.0 software (Axon Instruments, Union City, Calif.) to obtain gene expression ratios. Transformed data were normalized by LOWESS regression [Cell Mol Life Sci. 2007 February; 64(4): 458-78] and analyzed with GeneSpring GX 7.3 software program (Agilent Technologies Inc. USA). With the 1-color default normalization, (Per chip: normalize to a median or percentile and Per gene: normalize to median), GeneSpring GX first divides each raw intensity value by the median of the chip. Then each value is further divided by the median value of each gene across samples, resulting in the final normalized value.

The microarray analysis result for Ca²⁺ signaling pathway is shown in Table 1 below.

TABLE 1

| Gene_Symbol | Ratio (G/N) | P-value | GenBank_Acc | Gene Description |
|---|---|---|---|---|
| ITPR1 | 0.45 | 0.0228 | NM_002222 | Inositol 1,4,5-triphosphate receptor, type 1 |
| ITPR2 | 1.78 | 0.0487 | NM_002223 | Inositol 1,4,5-triphosphate receptor, type 2 |
| ITPR3 | 2.29 | 0.9399 | NM_002224 | Inositol 1,4,5-triphosphate receptor, type 3 |
| RYR1 | 0.66 | 0.0211 | NM_000540 | Ryanodine receptor 1 (skeletal) |
| RYR2 | 0.64 | 0.2622 | NM_001035 | Ryanodine receptor 2 (cardiac) |
| RYR3 | 0.91 | 0.7269 | NM_001036 | Ryanodine receptor 3 |
| TRPC1 | 0.66 | 0.0130 | NM_003304 | Transient receptor potential cation channel, subfamily C, member 1 |
| TRPC2 | 1.08 | 0.8419 | X89067 | Transient receptor potential cation channel, subfamily C, member 2 |
| TRPC3 | 0.91 | 0.8313 | NM_003306 | Transient receptor potential cation channel, subfamily C, member 3 |
| TRPC4 | 0.76 | 0.1015 | NM_016179 | Transient receptor potential cation channel, subfamily C, member 4 |
| TRPC5 | 0.71 | 0.4365 | NM_012471 | Transient receptor potential cation channel, subfamily C, member 5 |
| TRPC6 | 2.08 | 0.0070 | NM_004621 | Transient receptor potential cation channel, subfamily C, member 6 |
| TRPC7 | 0.89 | 0.5719 | NM_020389 | Transient receptor potential cation channel, subfamily C, member 7 |
| EGFR | 3.14 | 0.0561 | X00588 | Epidermal growth factor receptor |
| F2R | 7.67 | 0.0083 | NM_001992 | Thrombin receptor PAR1 |
| BDKRB1 | 0.68 | 0.0661 | NM_000710 | Bradykinin receptor B1 |
| BDKRB2 | 0.62 | 0.0039 | NM_000623 | Bradykinin receptor B2 |
| ATP2A1 | 0.87 | 0.2609 | NM_173201 | ATPase, Ca++ transporting, cardiac muscle, fast twitch 1 |
| ATP2A2 | 1.11 | 0.5573 | NM_001681 | ATPase, Ca++ transporting, cardiac muscle, slow twitch 2 |
| ATP2A3 | 0.69 | 0.0551 | NM_174953 | ATPase, Ca++ transporting, ubiquitous |
| ATP2B1 | 0.83 | 0.6383 | NM_001682 | ATPase, Ca++ transporting, plasma membrane 1 |
| ATP2B2 | 0.68 | 0.0129 | NM_001001331 | ATPase, Ca++ transporting, plasma membrane 2 |
| ATP2B3 | 0.53 | 0.0103 | NM_021949 | ATPase, Ca++ transporting, plasma membrane 3 |
| ATP2B4 | 0.61 | 0.0046 | NM_001684 | ATPase, Ca++ transporting, plasma membrane 4 |
| ATP2C1 | 1.35 | 0.0162 | NM_014882 | ATPase, Ca++ transporting, type 2C, member 1 |
| PLCB1 | 0.54 | 0.00500 | NM_015192 | Phospholipase C, beta 1 |
| PLCB2 | 0.76 | 0.99616 | NM_004573 | Phospholipase C, beta 2 |
| PLCB3 | 1.35 | 0.15364 | NM_000932 | Phospholipase C, beta 3 |
| PLCB4 | 0.67 | 0.34884 | NM_000933 | Phospholipase C, beta 4 |
| PLCD1 | 0.82 | 0.71120 | NM_006225 | Phospholipase C, delta 1 |
| PLCD3 | 0.62 | 0.33271 | NM_133373 | Phospholipase C, delta 3 |
| PLCD4 | 0.9 | 0.44077 | NM_032726 | Phospholipase C, delta 4 |
| PLCE1 | 2.26 | 0.44776 | NM_016341 | Phospholipase C, epsilon 1 |
| PLCG1 | 1.41 | 0.01088 | NM_002660 | Phospholipase C, gamma 1 |
| PLCG2 | 1.4 | 0.25410 | NM_002661 | Phospholipase C, gamma 2 |
| PLCH2 | 0.66 | 0.25410 | BC043358 | Phospholipase C, eta 2 |
| ITPK1 | 0.66 | 0.1666 | NM_014216 | Inositol 1,3,4-triphosphate 6/6 kinase |
| ITPKA | 0.85 | 0.00306 | NM_002220 | Inositol 1,4,6-trisphosphate 3-kinase A |
| ITPKB | 0.79 | 0.81113 | NM_002221 | Inositol 1,4,6-trisphosphate 3-kinase B |
| ITPKC | 0.79 | 0.57628 | NM_025194 | Inositol 1,4,5-trisphosphate 3-kinase C |

Table 1 shows numerical values representing degrees of expression (indicated in figures) of genes associated with CA²⁺ involving signaling system that is found to be targeted by caffeine. It was observed that expression levels of genes such as ITPR3, TRPC6, EGFR, F2R, PLCE1, and the like were significantly increased.

What is claimed is:

1. A method for treatment of a brain tumor, comprising administering a therapeutically effective amount of at least one selected from the group consisting of caffeine, 7-isopropyl theophylline, 7-(β-hydroxyethyl)theophylline, xanthine, theophylline, 1,7-dimethyl-3-isobutyl xanthine, and their pharmaceutically acceptable salts, to a patient in need thereof.

2. The method according to claim 1, wherein the brain tumor is one or more selected from the group consisting of glioma, meningioma, pituitary adenoma, schwannoma, craniopharyngioma, and congenital brain tumor.

3. The method according to claim 2, wherein the glioma is glioblastoma.

4. The method according to claim 1, wherein the treatment of a brain tumor is by selectively blocking inositol-1,4,5-triphospate receptor subtype 3 (IP₃R3) on lesion cells.


SEQUENCE LISTING (8 sequences)

SEQ ID NO: 1 - DNA, 17 bp, Artificial Sequence
  IP3R1 sense primer
ctctgatcgtttacctg

SEQ ID NO: 2 - DNA, 20 bp, Artificial Sequence
  ITPR1 antisense primer
tcttctgcttctcactcctc

SEQ ID NO: 3 - DNA, 20 bp, Artificial Sequence
  IP3R2 sense primer
agaaggagtttggagaggac

SEQ ID NO: 4 - DNA, 20 bp, Artificial Sequence
  IP3R2 antisense primer
tcaccacctttcacttgact

SEQ ID NO: 5 - DNA, 20 bp, Artificial Sequence
  IP3R3 sense primer
ctgttcaacgtcatcaagag

SEQ ID NO: 6 - DNA, 20 bp, Artificial Sequence
  IP3R3 antisense primer
catcaacagagtgtcacagg

SEQ ID NO: 7 - DNA, 20 bp, Artificial Sequence
  GAPDH sense primer
agctgaacgggaagctcact

SEQ ID NO: 8 - DNA, 20 bp, Artificial Sequence
  GAPDH antisense primer
tgctgtagccaaattcgttg